(19) United States
(12) Patent Application Publication
(10) Pub. No.: US 2012/0302840 A1
(43) Pub. Date: Nov. 29, 2012

(54) BIOLOGICAL INFORMATION MEASUREMENT APPARATUS

(75) Inventor: Nobuo KUBO, Kyoto-shi (JP)

(73) Assignee: OMRON HEALTHCARE CO., LTD., Muko-shi (JP)

(21) Appl. No.: 13/571,063

(22) Filed: Aug. 9, 2012

Related U.S. Application Data
(63) Continuation of application No. PCT/JP2011/050348, filed on Jan. 12, 2011.

(30) Foreign Application Priority Data
Mar. 11, 2010 (JP) 2010-054401

(51) Int. Cl. A61B 5/00

(52) U.S. Cl. 600/300

(57) ABSTRACT

When an operation for causing a healthcare device to transfer a measurement value is performed on a communication apparatus, the communication apparatus issues a transmission request to the healthcare device. The healthcare device is capable of measuring and storing biological information on a plurality of subjects, and in response to the request, associates untransmitted measurement values for all of the subjects with memory IDs of storage regions corresponding to the respective subjects and storing the measurement values and a serial number of the healthcare device and collectively transmits the measurement values, the data on which is transferred to a server. In the server, a storage region is specified for each measurement value based on the memory ID and the serial number associated with the measurement value, and the measurement value is stored in the specified region.

### TECHNICAL FIELD

The present invention relates to a biological information measurement apparatus, a biological information management apparatus, a biological information management system, a biological information management program, and a method of managing biological information, and particularly relates to a biological information measurement apparatus, a biological information management apparatus, a biological information management system, a biological information management program, and a method of managing biological information that measure and manage biological information on a plurality of subjects.

### BACKGROUND ART

Some of healthcare devices such as body composition monitors with scale and blood pressure monitors can store a measurement value and transfer the stored measurement value to the outside in response to a predetermined operation. In this case, by applying, for example, a technology that is disclosed in JP 2007-318289A (hereinafter referred to as Patent Literature 1) and that transmits data to a server along with a terminal ID of a wireless terminal, it is possible to transmit characteristic information (device ID) of a healthcare device to a server along with a measurement value, thereby accumulating and managing the measurement value on the server side based on the device ID.

### CITATION LIST

### Patent Literature

Patent Literature 1: JP 2007-318289A

### SUMMARY OF INVENTION

### Technical Problem

With regard to healthcare devices, cases where a healthcare device is used by a plurality of subjects, for example, cases where a healthcare device is placed in a household and used for measurement by a plurality of members of that household, are conceivable. In such cases, measurement values for the plurality of subjects are sorted by subjects and stored in the healthcare device.

In cases where a healthcare device like this transmits a measurement value to a server for data management, it is necessary that data in which a subject is specified be transmitted. For this reason, if a mistake is made in an operation for specifying a subject (for example, selection of a user number) during data transmission, a problem arises in that the measurement value is no longer appropriately sorted and stored on the server side.

Moreover, it is necessary that an operation for transmitting data be performed for each subject, and there also is a problem in that the operation for transmitting data is complicated.

Also, since it is necessary that the operation for transmitting data be performed for each subject, there is a problem in that an operation for transmitting data for all of the subjects is complicated. Moreover, the plurality of subjects, for example, members of a family may include a member inexperienced in operating a computer or a mobile phone, and there is a problem in that it is very difficult for that member to perform the operation for transmitting data.

Furthermore, in cases where the technology disclosed in Patent Literature I is applied to a healthcare device, it is assumed that measurement for a single subject is performed on a single healthcare device, and if a single healthcare device is used by a plurality of subjects, there is a problem in that data can no longer be appropriately sorted and stored on the server side because the subjects cannot be identified in each piece of data.

The present invention has been achieved in light of such problems, and an object thereof is to provide a biological information measurement apparatus, a biological information management apparatus, a biological information management system, a biological information management program, and a method of managing biological information that are capable of appropriately measuring and managing biological information on a plurality of subjects.

### Solution to Problem

In order to achieve the aforementioned object, according to one aspect of the present invention, a biological information measurement apparatus is capable of measuring biological information on a plurality of subjects and storing the biological information on a subject-by-subject basis, and includes a designation unit for designating a subject, a measurement unit for measuring biological information on the subject, a storage unit for storing the subject and a measurement value obtained by the measurement unit in association with each other, an accepting unit for accepting an instruction to transmit a measurement value stored in the storage unit, a communication unit for transmitting a measurement value stored in the storage unit to another apparatus, and a control unit for performing a process in which when the accepting unit accepts an instruction from a user who is designated as one of the plurality of subjects stored to transmit a measurement value associated with the user, the measurement value for the user and measurement values for subjects other than the user of the plurality of subjects stored in the storage unit are each associated with information corresponding to the subject whom the measurement value is associated with and information characteristic of the biological information measurement apparatus, and the communication unit collectively transmits the measurement values to the other apparatus.

Preferably, when the accepting unit accepts the instruction from the user, the control unit causes all of the measurement values for the user and the subjects other than the user stored in the storage unit to be transmitted to the other apparatus, and deletes the measurement values transmitted by the communication unit from the storage unit in response to a notification from the other apparatus that has received the measurement values.

Preferably, the measurement values stored in the storage unit are each further associated with information distinguishing whether or not the measurement value has already been transmitted by the communication unit, and when the accepting unit accepts the instruction from the user, the control unit transmits a measurement value classified as a measurement value that has not yet been transmitted by the communication unit, of the measurement values for the user and the subjects other than the user stored in the storage unit, to the other apparatus.

More preferably, in response to a notification from the other apparatus that has received the measurement value, the control unit updates the information associated with the measurement value in the storage unit and distinguishing whether or not the measurement value has already been transmitted.

Preferably, the accepting unit includes a selection unit for selecting whether to collectively transmit measurement values for all of the plurality of subjects stored in the storage unit including the user or to designate, for each of the subjects other than the user of the plurality of subjects, whether or not to collectively transmit measurement values for the subject.

According to another aspect of the present invention, a biological information management apparatus is an apparatus for managing biological information measured on a biological information measurement apparatus, and includes a communication unit for receiving a measurement value from the biological information measurement apparatus, the measurement value being associated with information designating a storage region of the measurement value in the biological information measurement apparatus and characteristic information of the biological information measurement apparatus, a storage unit for storing a correspondence relationship between a region in a storage apparatus and a combination of the information designating a storage region in the biological information measurement apparatus and the characteristic information of the biological information measurement apparatus, and a control unit for storing the measurement value in the region in the storage apparatus specified from the combination of the information designating a storage region and the characteristic information of the biological information measurement apparatus, wherein when a plurality of measurement values associated with combinations of the information designating different storage regions and the characteristic information of the biological information measurement apparatus are received, the control unit stores each of the plurality of measurement values received in the region in the storage apparatus specified by the combination associated with the measurement value.

Preferably, the biological information management apparatus further includes a first notification unit for issuing, when the control unit stores the measurement value, a notification to that effect.

Preferably, the biological information management apparatus further includes a second notification unit for issuing, if the control unit cannot specify a region in the storage apparatus from the combination associated with the measurement value, a notification to that effect.

Preferably, the biological information management apparatus further includes an updating unit for updating the correspondence relationship between a region in the storage apparatus and a combination of the information designating a storage region in the biological information measurement apparatus and the characteristic information of the biological information measurement apparatus, the correspondence relationship being stored in the storage unit.

Preferably, the storage unit further stores a correspondence relationship between a subject and a region in the storage apparatus, the biological information management apparatus further includes an accepting unit for accepting a viewing request with respect to a measurement value for the subject, and the control unit further executes a process for reading out the requested measurement value for the subject from the region in the storage apparatus corresponding to the subject and transmitting the measurement value.

More preferably, the storage unit stores information for each subject, the information specifying a user who is permitted to view a measurement value for the subject, and if it is determined, based on information specifying the user contained in the viewing request, that the user is permitted to view a measurement value for the subject, the control unit transmits the measurement value for the subject.

Preferably, the storage unit further stores a correspondence relationship between a subject and a region in the storage apparatus, and the biological information management apparatus further includes a third notification unit for issuing, when the control unit specifies a subject with respect to whom a period of time that has elapsed from time and date of measurement of a latest measurement value stored to the present time is longer than a predefined threshold, a notification to that effect to a destination associated with the specified subject.

According to still another aspect of the present invention, a biological information management system is a system including a biological information measurement apparatus and a biological information management apparatus. The biological information measurement apparatus is capable of measuring biological information on a plurality of subjects and storing the biological information on a subject-by-subject basis, and includes a designation unit for designating a subject, a measurement unit for measuring biological information on the subject, a storage unit for storing the subject and a measurement value obtained by the measurement unit in association with each other, an accepting unit for accepting an instruction to transmit a measurement value stored in the storage unit, a communication unit for transmitting a measurement value stored in the storage unit to the biological information management apparatus, and a control unit for performing a process in which when the accepting unit accepts an instruction from a user designated as one of the plurality of subjects stored to transmit a measurement value associated with the user, the measurement value for the user and measurement values for subjects other than the user of the plurality of subjects stored in the storage unit are each associated with information corresponding to the subject whom the measurement value is associated with and information characteristic of the biological information measurement apparatus, and the communication unit collectively transmits the measurement values to another apparatus. The biological information management apparatus is a biological information management apparatus for managing biological information measured on the biological information measurement apparatus, and includes a communication unit for receiving a measurement value from the biological information measurement apparatus, the measurement value being associated with information, as the information corresponding to the subject, designating a storage region of the measurement value in the biological information measurement apparatus and the characteristic information of the biological information measurement apparatus, a storage unit for storing a correspondence relationship between a region in a storage apparatus and a combination of the information designating a storage region and the characteristic information of the biological information measurement apparatus, and a control unit for storing the measurement value in the region in the storage apparatus specified from the combination of the information designating a storage region and the characteristic information of the biological information measurement apparatus, and when a plurality of measurement values associated with combinations of the information designating different storage regions and the characteristic information of the biological information measurement apparatus are received from the biological information measurement apparatus, the control unit of the biological information management apparatus stores each of the plurality of measurement values received in the region in the storage apparatus specified by the combination associated with the measurement value.

According to still another aspect of the present invention, a biological information management program is a program for causing a computer to execute a process for managing biological information measured on a biological information measurement apparatus, the process including the steps of receiving a measurement value from the biological information measurement apparatus, the measurement value being associated with information designating a storage region of the measurement value in the biological information measurement apparatus and characteristic information of the biological information measurement apparatus, specifying a region in a storage apparatus from a combination of the information designating a storage region in the biological information measurement apparatus and the characteristic information of the biological information measurement apparatus, and storing the measurement value in the specified region in the storage apparatus, wherein when a plurality of measurement values associated with combinations of the information designating different storage regions and the characteristic information of the biological information measurement apparatus have been received from the biological information measurement apparatus in the measurement value receiving step, each of the plurality of measurement values received is stored in the region in the storage apparatus specified by the combination associated with the measurement value in the storing step.

According to still another aspect of the present invention, a method of managing biological information is a method of managing a measurement value obtained by a biological information measurement apparatus by storing the measurement value in a storage apparatus, the biological information measurement apparatus being capable of measuring biological information on a plurality of subjects and storing the biological information on a subject-by-subject basis, the method including a step in which the biological information measurement apparatus accepts an instruction to transmit a stored measurement value, a step in which when an instruction from a user designated as one of the plurality of subjects stored in the biological information measurement apparatus to transmit a measurement value associated with the user is accepted, the measurement value for the user and measurement values for subjects other than the user of the plurality of subjects stored in the biological information measurement apparatus are each associated with information designating a storage region in the biological information measurement apparatus, the storage region corresponding to the subject associated with the measurement value, and information characteristic of the biological information measurement apparatus, and the measurement values are collectively transmitted, a step in which for each of the plurality of measurement values transmitted from the biological information measurement apparatus and associated with combinations of the information designating different storage regions in the biological information measurement apparatus and the characteristic information of the biological information measurement apparatus, a region in the storage apparatus is specified from the combination associated with the measurement value, and a step in which each of the plurality of measurement values transmitted is stored in the region in the storage apparatus specified by the combination associated therewith.

### Advantageous Effects of Invention

According to the present invention, the operation for transferring data from a healthcare device can be facilitated, and data management can be appropriately performed.

### BRIEF DESCRIPTION OF DRAWINGS

FIG. 1 is a diagram showing a specific example of the configuration of a biological information management system according to an embodiment and a specific example of the configuration of apparatuses included in the biological information management system.

FIG. 2 is a diagram showing a specific example of the manner in which measurement values are stored in a healthcare device.

FIG. 3 is a flowchart showing the flow of a process for registering information regarding a subject with the biological information management system.

FIG. 4 is a diagram showing a specific example of a login screen.

FIG. 5 is a diagram showing a specific example of user data stored in a server.

FIG. 6 is a flowchart showing the flow of a process for transferring a measurement value in the biological information management system.

FIG. 7A is a diagram showing a specific example of a screen for instructing data transfer.

FIG. 7B is a diagram showing a specific example of a screen for instructing data transfer.

FIG. 8 is a diagram showing a specific example of a screen for warning that a transferred measurement value is a measurement value for a subject who is not registered with the biological information management system.

FIG. 9 is a diagram showing a specific example of a screen that appears when a measurement value stored in the server is to be viewed.

FIG. 10 is a diagram showing a specific example of a screen for notifying that no measurement value has been transferred for a predetermined period of time.

### DESCRIPTION OF EMBODIMENTS

The following describes an embodiment of the present invention with reference to the drawings. In the following description, parts and constituent elements that are the same will be given the same reference signs. The names and functions thereof are also the same.

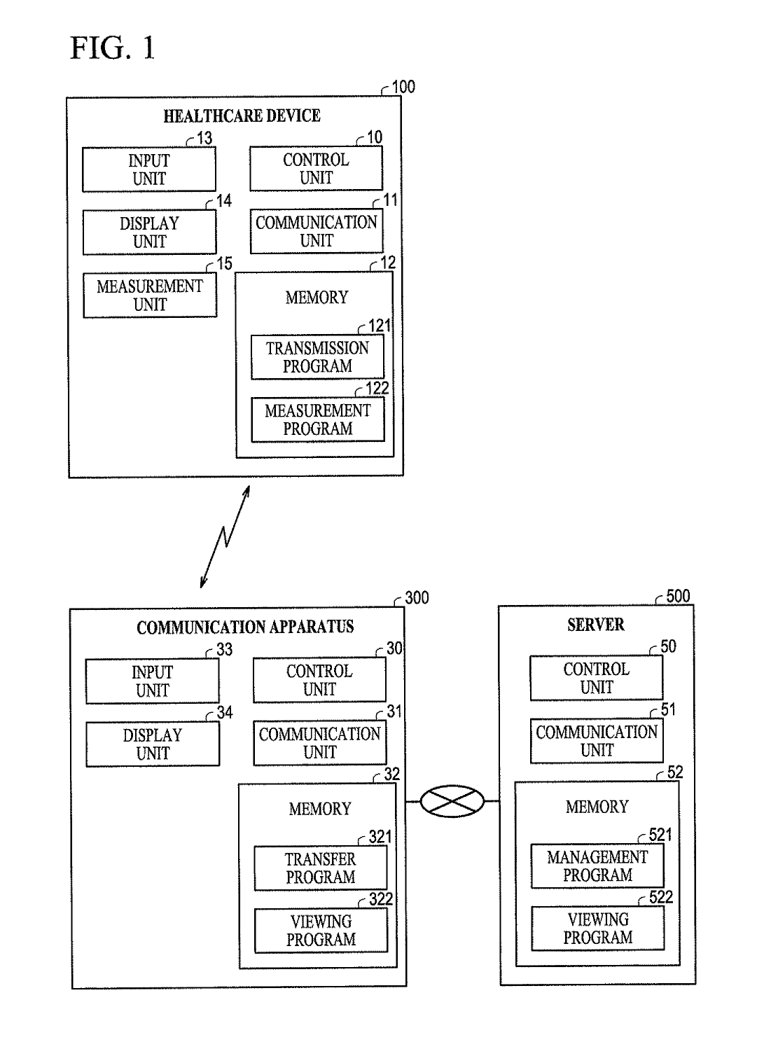

Referring to FIG. 1, a biological information management system (hereinafter referred to as a system) of the present embodiment includes a healthcare device 100, which is, for example, a body composition monitor with scale or a blood pressure monitor, serving as a biological information measurement apparatus, a communication apparatus 300 communicable with the healthcare device 100, and a server 500 communicable with the communication apparatus 300. A common personal computer (hereinafter also referred to as a PC) equipped with a communication function, a mobile phone handset, or the like corresponds to the communication apparatus 300. Alternatively, a dedicated communication apparatus such as a set-top box may be used as the communication apparatus 300.

The healthcare device 100 and the communication apparatus 300 may perform wireless communication such as infrared communication, or may perform wired communication through a dedicated communication cable connected therebetween. The communication apparatus 300 and the server 500 may perform communication over the Internet, may perform wireless communication such as infrared communication, or may perform wired communication through a dedicated communication cable connected therebetween. In the description here, it is assumed that the healthcare device 100 and the communication apparatus 300 perform wireless communication, and the communication apparatus 300 and the server 500 perform communication over the Internet.

Furthermore, referring to FIG. 1, the healthcare device 100 includes a control unit 10 including a CPU (Central Processing Unit) and intended to perform overall control, a communication unit 11 for communicating with the communication apparatus 300, a memory 12 for storing a program executed by the control unit 10, a measurement value, and identification information (hereinafter referred to as a serial number) characteristic of the healthcare device 100, an input unit 13 such as an operating button, a display unit 14, and a measurement unit 15 for measuring biological information such as the blood pressure value, the weight, and the percentages of the components of body composition. The input unit 13 includes a measurement button and also a button for designating a subject. The program stored in the memory 12 as the program executed by the control unit 10 includes a transmission program 121 for causing an operation for transmitting a measurement value to be executed and a measurement program 122 for causing a measuring operation to be executed.

When the measuring operation is performed in the healthcare device 100, the control unit 10 reads out the measurement program 122 in accordance with an operation signal that is provided from the input unit 13 in response to an operation for starting the measurement, and outputs a control signal to the measurement unit 15 according to the execution of the program.

Those that are similar to a measurement unit provided in an ordinary body composition monitor with scale if the healthcare device 100 is a body composition monitor with scale, a measurement unit provided in an ordinary blood pressure monitor if the healthcare device 100 is a blood pressure monitor, or the like can be used as the measurement unit 15. The measurement unit 15 performs the measuring operation in accordance with the control signal from the control unit 10 and returns a signal indicating the result of the measuring operation to the control unit 10.

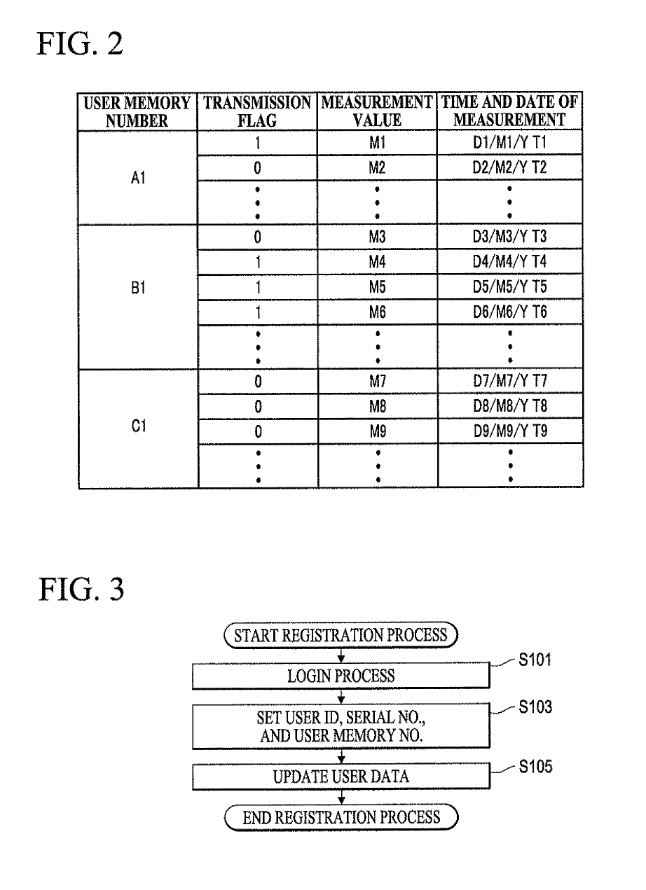

The control unit 10 converts the signal from the measurement unit 15 to a measurement value in accordance with the measurement program 122, and stores the measurement value in a predetermined region in the memory 12. At this time, the control unit 10 associates the measurement value with information specifying the time and date of measurement and stores the measurement value on a subject-by-subject basis with each subject being specified by an operation signal based on an operation of selecting the subject from the input unit 13. As a specific example, as shown in FIG. 2, memory regions related to previously registered subjects are set in the memory 12, and the control unit 10 stores a measurement value and the information specifying the time and date of measurement for each subject in the corresponding memory region. FIG. 2 shows an example in which user memory numbers serving as characteristic information are assigned to the memory regions related to the subjects and an example in which for each user memory number, measurement values and the information specifying the time and date of measurement for the corresponding subject are stored. Furthermore, as shown in FIG. 2, each measurement value may be provided with a transmission flag indicating whether the measurement value has been transmitted by an operation for transmitting the measurement value, which will be described later, or has not yet been transmitted. In an example in FIG. 2, the flag of a measurement value that has been transmitted is on {the flag is set to 1), and the flag of a measurement value that has not yet been transmitted is off (the flag is set to 0).

When the operation for transmitting a measurement value is performed in the healthcare device 100, the control unit 10 reads out the transmission program 121, reads out necessary data from a predetermined region in the memory 12 according to the execution of the program, and passes the data to the communication unit 11 and causes the communication unit 11 to transmit the data. The operation for transmitting a measurement value will be described later.

The communication apparatus 300 includes a control unit 30 including a CPU and intended to perform overall control, a communication unit 31 for communicating with the healthcare device 100 in a wireless manner and communicating with the server 500 over the Internet, a memory 32 for storing, for example, a program executed by the control unit 30, an input unit 33 such as an operating button, and a display unit 34. The program stored in the memory 32 as the program executed by the control unit 30 includes a transfer program 321 for causing an operation for transferring a measurement value to be executed and a viewing program 322 for causing an operation for viewing a measurement value stored in the server 500 to be executed.

The server 500 includes a control unit 50 including a CPU and intended to perform overall control, a communication unit 51 for communicating with the communication apparatus 300 over the Internet, and a memory 52 for storing, for example, a program executed by the control unit 50. The program stored in the memory 52 as the program executed by the control unit 50 includes a management program 521 for causing an operation for managing a measurement value to be executed and a viewing program 522 for causing an operation for allowing a stored measurement value to be viewed on the communication apparatus 300 to be executed.

The memory 52 of the server 500 further includes regions for storing measurement values transmitted from the healthcare device 100. The regions are each made to correspond to a combination of a serial number and a user memory number associated with a measurement value. When a measurement value is received, the control unit 50 that executes the management program 521 specifies the region for storing the measurement value. In the following description, identification information characteristic of those regions will also be referred as database IDs.

In the present system, prior to the management of a measurement value, a process for registering information regarding a subject with the system, which is illustrated in the flowchart of FIG. 3, is performed. This process is started by a management program, which is not shown, stored in the memory 32 being executed by the control unit 30 as a result of the input unit 33 of the communication apparatus 300 being operated. In accordance with data that is transmitted according to the execution of this program, the control unit 50 of the server 500 executes the management program 521 stored in the memory 52.

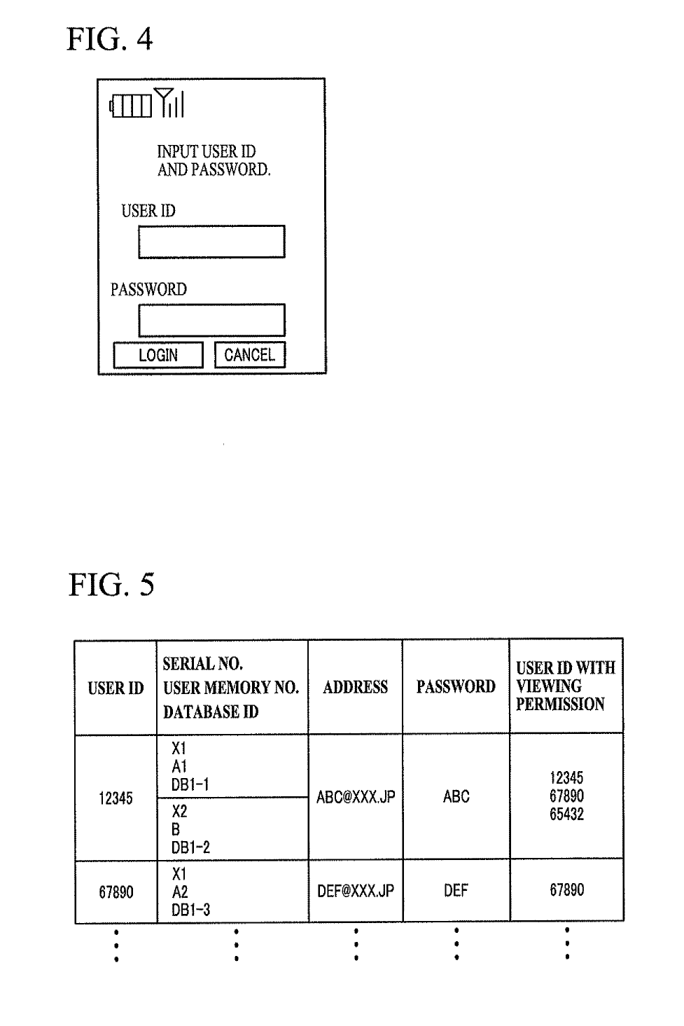

Referring to FIG. 3, when execution of the management program, which is not shown, is started by the control unit 30 of the communication apparatus 300, in step S101, a login screen for designating a user as one of the subjects, as shown in FIG. 4, is displayed on the display unit 34, and the input of identification information (user ID) of the subject and a password is accepted. The information input through the screen in FIG. 4 is sent from the communication unit 31 to the server 500, and checked against user data, which will be described later, by the control unit 50 of the server 500. If the user ID and the password match those registered in the user data, which will be described later, it is determined that the login is successful, and the control unit 30 of the communication apparatus 300 and the control unit 50 of the server 500 execute the following operations.

In the management program after the successful login, when an instruction to execute the registration process is accepted, the management program branches to a registration program, which is not shown. According to the execution of this program, in step S103, the control unit 30 accepts the input of a user ID, a serial number, and a user memory number to be registered. The control unit 30 may accept the information such as the user ID to be registered by causing the display unit 34 to display an input screen, which is not shown, and receiving an operation signal from the input unit 33, or may accept the information by receiving the information transmitted from the healthcare device 100. In the latter case, it is assumed that a registration operation is performed on the healthcare device 100, and the information is transmitted to the communication apparatus 300 in accordance with a registration program, which is not shown. The accepted information is transmitted to the server 500.

When the information such as the user ID has been received from the communication apparatus 300, in step S105, the control unit 50 of the server 500 updates the user data stored in the memory 52 and finishes the registration process.

Referring to FIG. 5, the memory 52 of the server 500 stores, as the user data for each subject, a user ID, a serial number and a user memory number and a database ID associated with these numbers, an address, which is a destination of a notification and the like, a login password, and a user ID of a subject whom the subject permits to view his/her measurement value or values. In the above-described step S105, the control unit 50 of the server 500 registers a new subject by adding the above-described newly accepted information to the user data in FIG. 5 as information regarding the new subject. It is also possible to alter or delete previously registered user data using the same method.

Note that as shown in FIG. 5, combinations of a plurality of serial numbers, user memory numbers, and database IDs associated with those numbers may be stored with respect to the same user ID. It is conceivable that this applies in the case where the same subject measures his/her biological information using a plurality of healthcare devices and transmits measurement values on all of the healthcare devices to the server 500.

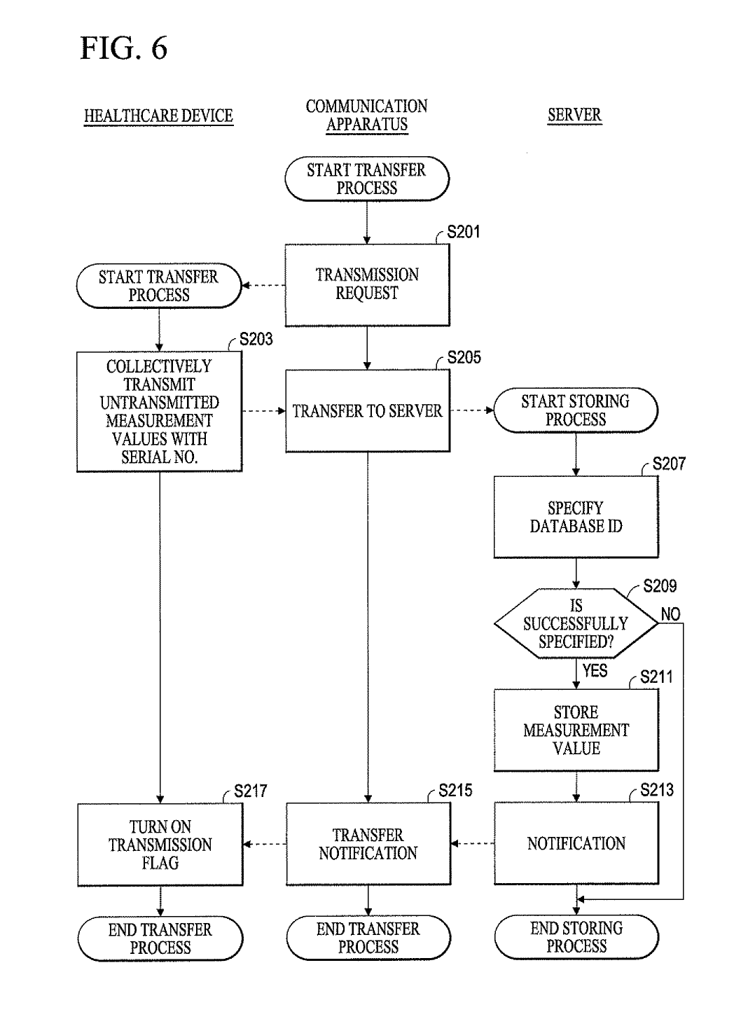

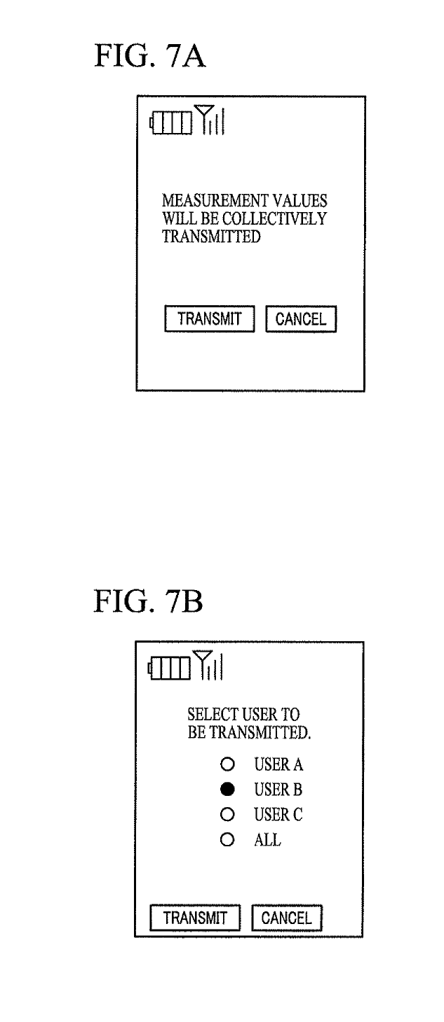

In the present system, a measurement value measured on the healthcare device 100 for a registered subject is transmitted to the server 500 by a predetermined operation and stored in the server 500. An example of transfer processes performed in the apparatuses at this time will be described using FIG. 6. The transfer processes illustrated in the flowchart of FIG. 6 are realized by the control units of the respective apparatuses executing the program stored in the memory. The processes in FIG. 6 are started as a result of a user who has been designated as one of the subjects by logging in using the login screen in FIG. 4 displayed on the display unit 34 of the communication apparatus 300 inputting a data transfer instruction to the communication apparatus 300 through a screen for instructing the transfer as shown in FIG. 7A.

Referring to FIG. 6, when the management program, which is not shown, executed by the control unit 30 of the communication apparatus 300 branches to the transfer program 321 and the transfer process is started, the above-described instruction is accepted, and thus a signal requesting the healthcare device 100 to transmit a predefined measurement value is output in step S201.

When the communication unit 11 has received the above-described signal, the control unit 10 of the healthcare device 100 executes the transmission program 121, thereby extracting measurement values that have not yet been transmitted out of measurement values stored in the memory 12, automatically associates each of the extracted measurement values with a user memory number indicating the memory region in which the measurement value is stored and a serial number of the healthcare device 100, and transmits the measurement values to the communication apparatus 300. The communication apparatus 300 that has received the measurement values from the healthcare device 100 transfers the measurement values to the server 500 in step S205.

Note that although the example in FIG. 6 is based on the assumption that the measurement values from the healthcare device 100 are transmitted to the server 500 via the communication apparatus 300, it is also possible that the healthcare device 100 is communicable with the server 500, the healthcare device 100 stores the address of the server 500 in advance, and the healthcare device 100 directly transmits the measurement values to the server 500.

Furthermore, in the example in FIG. 6, the communication apparatus 300 accepts the instruction to transfer data and issues the transmission request to the healthcare device 100, but if the input unit 13 of the healthcare device 100 includes an input unit for instructing data transfer, such as a transfer button, it is also possible that the transfer process is started based on an operation with the transfer button by the subject who has been designated using the button for designating a subject, and the healthcare device 100 transmits the measurement values in response to an operation signal resulting from that operation (with no need for the transmission request from the communication apparatus 300). Alternatively, the transfer process is started not only by the control unit 10 accepting the operation for transfer through the transfer button or the like, but the detection of a predefined event or timing, for example, the detection of insertion of a storage medium such as a USB (Universal Serial Bus) by a storage medium reading unit, which is not shown, of the healthcare device 100, the timing at which an ordinary measuring operation is finished, or the like may act as a trigger, automatically starting the transfer process. It is to be construed that the acceptance of the transmission request by the control unit 10 also includes the detection by the control unit 10 as described above.

Moreover, in the example in FIG. 6, an example is illustrated in which, in response to the data transfer instruction through the screen in FIG. 7A, untransmitted measurement values for not only the user, that is, the subject who has instructed the transfer but also other subjects of the plurality of subjects stored are collectively transferred. In the present system, preferably, a single transfer operation from the user who has logged in (or the user designated as one of the subjects) allows untransmitted measurement values for not only the user but all of the subjects stored to be collectively transferred to the server 500 without the need to individually designate the subjects. In this manner, untransmitted measurement values for all of the subjects stored are collectively transmitted, so that in cases where a plurality of subjects measure their biological information such as the blood pressure value and the weight using the healthcare device 100, the need to perform the data transfer operation for each subject, the operation for designating a subject, the operation for designating a measurement value to be transferred, or the like is eliminated, and the operation for transferring data can be significantly facilitated. Moreover, when compared with cases where the data transfer operation is performed for each subject or for each measurement value, the traffic necessary for data transfer can be suppressed.

As another example, in cases where the healthcare device 100 stores measurement values for a plurality of subjects, for each of the subjects other than the user who has logged in, whether or not to collectively transfer untransmitted measurement values for the subject may be designated using a screen as in FIG. 7B. Moreover, it is also possible that whether to transfer measurement values on a subject-by-subject basis or to transfer measurement values for all of the subjects stored may be selected. The transfer request from the communication apparatus 300 at this time contains information designating a target subject of transfer, and the control unit 10 of the healthcare device 100 transmits untransmitted measurement values for the designated subject. Moreover, in the case where the data transfer is instructed on the healthcare device 100 as described above, it is possible to designate a target subject of transfer using the button for designating a subject included in the input unit 13 and afterward instruct the transfer by, for example, pressing the transfer button.

In any example, the healthcare device 100 automatically associates a measurement value to be transmitted with the user memory number indicating the memory region in which the measurement value is stored and the serial number of the healthcare device 100, with no need for an operation by an operator, so that no mistake is made in designating a subject for each measurement value, and the measurement values can be appropriately stored in the server.

When the communication unit 51 has received a measurement value from the healthcare device 100, the control unit 50 of the server 500 executes the management program 521, thereby specifying a database ID based on the user memory number and the serial number associated with the received measurement value in step S207. Here, the corresponding database ID can be specified by, for example, scanning the user data in FIG. 5.

In this manner, the healthcare device 100 transmits a measurement value associated with information specifying a subject, so that the server 500 can sort and store the measurement value based on that information, and the measurement value can be appropriately stored in the server. Moreover, during a data transfer operation, there is no need to designate a subject for each measurement value, so that the operation is facilitated, and also no mistake is made in designating a subject, so that the measurement values can be appropriately stored in the server.

If the database ID is successfully specified based on the user memory number and the serial number (YES in step S209), the transmitted measurement value is a measurement value for a subject who is already registered, and therefore, in step S211, the control unit 50 stores the received measurement value associated with the information specifying the time and date of measurement in the specified region in the memory 52.

If the database ID is not successfully specified (NO in step S209), the transmitted measurement value is not a measurement value for a registered subject, and therefore the control unit 50 does not perform the storing operation in step S211.

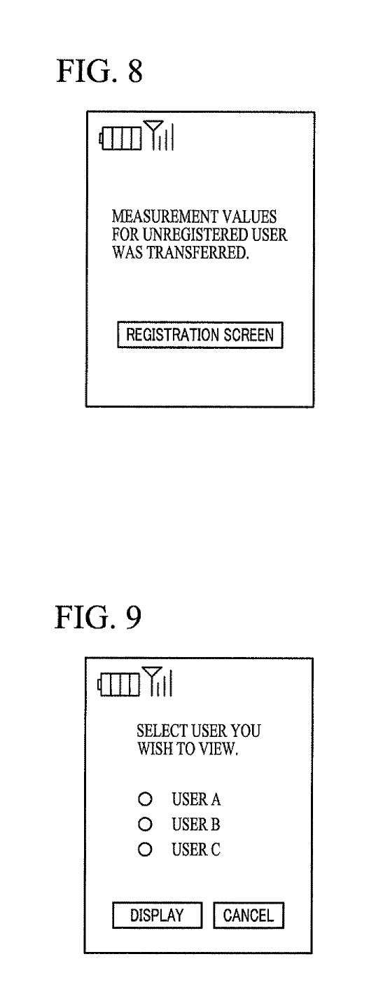

At this time, a screen as shown in FIG. 8, which warns that the measurement value is a measurement value for an unregistered subject, may be displayed on the communication apparatus 300 by the server 500 informing the communication apparatus 300 to that effect. Furthermore, as illustrated in FIG. 8, it is also possible that a button or the like for instructing to start the registration process is provided on the warning screen, and the start of the process for registering the unregistered subject is directly accepted after the warning is displayed.

The transfer process of transferring and storing a measurement value in the server may be finished now. However, preferably, the server 500, which has stored the measurement value, transmits a notification to the communication apparatus 300 to that effect in step S213. The communication apparatus 300 that has received this notification outputs a signal to the healthcare device 100 to that effect in step S215. In the healthcare device 100 that has received the signal, in step S217, the control unit 10 sets the transmission flag for the transmitted measurement value of the measurement values stored in the memory 12 so as to indicate that the measurement value has been transmitted.

Note that, also here, as is the case with the transmission of a measurement value, the server 500 may transmit a signal to the healthcare device 100 to the effect that storage of the measurement value has been completed.

Moreover, although the above-described example is based on the assumption that the memory 12 of the healthcare device 100 stores all the measurement values, each of which is provided with a transmission flag indicating that it has been transmitted or has not yet been transmitted, when the healthcare device 100 has transmitted a measurement value and received a signal to the effect that the storage thereof is completed, the relevant measurement value may be deleted from the memory 12, or may be moved to a predetermined memory region and deleted after a predetermined period of time has elapsed.

It goes without saying that at the point in time when the transmission is completed in step S203 above, the control unit 10 may set the transmission flag so as to indicate that the measurement value has been transmitted or may delete the measurement value from the memory 12. However, notifying the completion of the storage as described above enables the healthcare device 100 to confirm that the storage of the measurement value into the server 500 has been completed before determining that the transmission has been completed, so that the measurement value can be reliably managed.

In this manner, the healthcare device 100 classifies the measurement values into transmitted measurement values and untransmitted measurement values as described above. Therefore, in cases where a measurement value that has already been transmitted is also stored in the memory 12, when transmitting a measurement value to the server 500, there is no need to perform the operation of designating the measurement value out of the stored measurement values, so that the operation for data transfer can be facilitated.

In the foregoing description and the exemplary screens, an example in which the communication apparatus 300 is a mobile phone handset has been described. In cases where the communication apparatus 300 is a mobile phone handset, the transfer program 321 and the like stored in the memory 32 may be stored in advance, may be downloaded by accessing a predetermined server (e.g., the server 500) in response to an operation, or may be realized by connecting to a predetermined Web page and executing a program of a server storing that Web page. This holds true in cases where the communication apparatus 300 is another device such as a PC.

The measurement value stored in the server 500 may be used in various ways by a function of the server 500 or by being transmitted to another apparatus and processed by that apparatus. An example is a process for viewing the measurement value stored in the server 500. The stored measurement value can be viewed on the server 500 itself and other apparatuses that are communicable with the server 500. The following description is based on the assumption that the measurement value is viewed on the communication apparatus 300.

In this case, the user who wishes to view a measurement value logs in from the login screen in FIG. 4 displayed on the display unit 34 as a result of the management program, which is not shown, being executed. When the control unit 30 accepts an instruction to execute a viewing process in the management program after the successful login to the server 500, the management program branches to the viewing program 322. According to the execution of that program, the control unit 30 passes information on the user who wishes to view the measurement value to the server 500, and issues a viewing request thereto.

In the server 500 that has received the viewing request, the control unit 50 executes the viewing program 522, scans the user data according to that program, and specifies a subject whose measurement value or values the user who has logged in is permitted to view. Information on the specified subject is transmitted to the communication apparatus 300.

In the communication apparatus 300, based on the received information on the subject, as shown in FIG. 9, viewable subjects whose measurement value or values can be viewed are presented on the display unit 34 in a selectable manner. Note that here, in addition to the selection of a subject, designation of the time and date of measurement that the user wishes to view or designation of the measurement period may also be accepted. When the designation of the subject whose measurement value or values the user wishes to view has been accepted through the screen in FIG. 9, the control unit 30 passes the information on that subject to the server 500 and issues a viewing request thereto. At this time, the control unit 30 may also pass information such as the time and date of measurement or the measurement period.

In the server 500 that has accepted the viewing request, the control unit 50 scans the user data and specifies the region in the memory 52 storing a measurement value or values for the subject, and transmits the measurement value or values stored in that region to the communication apparatus 300. If the time and date of measurement or the measurement period is designated, the control unit 50 scans the associated time and date of measurement and extracts a relevant measurement value or values, and then transmits the extracted measurement value or values to the communication apparatus 300. The measurement value or values received are displayed by the communication apparatus 300, and thus the user can view the measurement value or values for the subject whom the user has designated.

Instead of the viewing process as described above, the server 500 may receive the information designating the subject whose measurement value or values the user wishes to view from the communication apparatus 300. In this case, the control unit 50 scans the user data and determines whether or not the user who wishes to view the measurement value or values (the user who has logged in) is permitted to view the measurement value or values for that particular subject. As a result, if the user is permitted to view the measurement value or values, the control unit 50 extracts and transmits the relevant measurement value or values to the communication apparatus 300, while if the user is not permitted, the control unit 50 notifies the communication apparatus 300 to that effect.

Since processing as described above is performed in the present system, measurement values stored in the server 500 can be viewed by an easy operation, and since a user with viewing permission is designated in the user data beforehand, it is possible to prohibit a user without permission from viewing. Therefore, both of the operability and the protection of personal information can be achieved.

Moreover, as illustrated in FIG. 5, the server 500 stores, as user data, the correspondence between a subject and a database ID, and it is assumed that in the user data, a plurality of database IDs may correspond to the same subject. Thus, even in cases where the same subject measures his/her biological information using a plurality of healthcare devices, measurement values for a designated subject can be displayed irrespective of the healthcare devices used for measurement.

Another example of the use of the measurement values stored in the server 500 is a process of prompting measurement value transmission. It is assumed that this process is performed in the server 500 at predetermined time intervals, at the timing at which an inquiry is received from the communication apparatus 300, at the timing at which a measurement value is transmitted from the healthcare device 100, and so on.

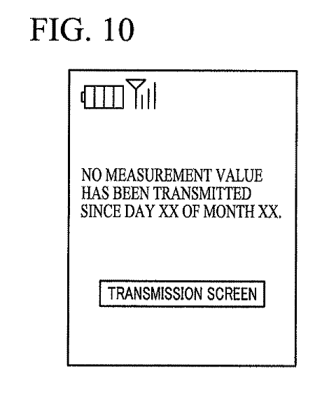

The server 500 calculates, at a predetermined timing, the period of time from the latest time and date of measurement to the present time for every subject registered in the user data and compares the calculated period of time with a predefined threshold. If the period of time from the latest time and date of measurement to the present time for any subject is longer than the threshold, the server 500 issues at least a notification to that effect to the address of the corresponding subject stored in the user data. Preferably, the notification includes the latest time and date of measurement of measurement values stored for this subject. This enables a communication device of this subject to display a notification screen shown in FIG. 10. More preferably, if the communication device of this subject is the communication apparatus 300, it is possible to provide a button or the like for accepting a data transfer instruction on the notification screen as shown in FIG. 10 and directly accept the start of a process for transferring the measurement values for the corresponding subject after the notification is displayed.

As a result of processing such as this being performed, transmission of the measurement values to the server 500 or measurement on the healthcare device 100 and transmission to the server 500 can be prompted. Furthermore, as illustrated in FIG. 5, the server 500 stores the correspondence between a subject and a database ID as user data, and in the user data, a plurality of database IDs may correspond to the same subject. Therefore, even if the same subject measures his/her biological information using a plurality of healthcare devices, the transfer status of measurement values for the designated subject can be checked irrespective of the healthcare devices used for measurement.

In the foregoing description, as shown in FIG. 1, it is assumed that the present system includes the healthcare device 100, the communication apparatus 300, and the server 500, but it is also possible that the present system does not include the communication apparatus 300, and communication is directly performed between the healthcare device 100 and the server 500. In this case, the healthcare device 100 may include all of the functions of the communication apparatus 300, or the server 500 may include all of the functions of the communication apparatus 300. Alternatively, the healthcare device 100 may include a part of the functions of the communication apparatus 300, and the server 500 include the other functions. Furthermore, the healthcare device 100 itself may function as the server 500 and store not only measurement values measured on the healthcare device 100 itself but also measurement values measured on other healthcare devices.

Furthermore, it is also possible to provide a management program, a viewing program, or the like for causing a computer to execute the measurement value transmission process, the viewing process, or the like of the present system described above. Such a program can also be recorded in a computer-readable recording medium such as a flexible disk attached to a computer, a CD-ROM (Compact Disk-Read Only Memory), a ROM (Read Only Memory), a RAM (Random Access

Memory), and a memory card and provided as a program product. Alternatively, the program can be recorded in a recording medium such as a hard disk built in a computer and provided. Moreover, the program can also be provided by downloading via a network.

Note that the above-described program may be a program that calls necessary modules among program modules provided as a part of an operating system (OS) of a computer in a predetermined sequence at predetermined timings and causes the modules to execute processing. In that case, the program itself does not include the modules and executes processing in cooperation with the OS. The above-described program may also include a program that does not contain a module such as this.

Moreover, the above-described program may be provided in a state in which it is incorporated in a part of another program. Also in this case, the program itself does not contain a module contained in the other program and executes processing in cooperation with the other program. The above-described program may also include a program such as this incorporated in another program.

The program product that is provided is installed to a program storage unit such as a hard disk and executed. Note that the program product includes the program itself and a recording medium in which the program is recorded.

The embodiment disclosed herein is to be considered in all respects as illustrative and not restrictive. The scope of the present invention is defined by the appended claims rather than by the description above, and all changes that fall within the meaning and scope equivalent to those of the claims are intended to be embraced therein.

### REFERENCE SIGNS LIST

10 control unit

11 communication unit
12 memory
13 input unit
14 display unit
15 measurement unit
30 control unit
31 communication unit
32 memory
33 input unit
34 display unit
50 control unit
51 communication unit
52 memory
100 healthcare device
121 transmission program
122 measurement program
300 communication apparatus
321 transfer program
322 viewing program
500 server
521 management program
522 viewing program

1. A biological information measurement apparatus capable of measuring biological information on a plurality of subjects and storing the biological information on a subject-by-subject basis, comprising:
a designation unit for designating a subject;
a measurement unit for measuring biological information on the subject;
a storage unit for storing the subject and a measurement value obtained by the measurement unit in association with each other;
an accepting unit for accepting an instruction to transmit a measurement value stored in the storage unit;
a communication unit for transmitting a measurement value stored in the storage unit to another apparatus; and
a control unit for performing a process in which when the accepting unit accepts an instruction from a user who is designated as one of the plurality of subjects to transmit a measurement value associated with the user, the measurement value for the user and measurement values for subjects other than the user of the plurality of subjects stored in the storage unit are each associated with information corresponding to the subject whom the measurement value is associated with and information characteristic of the biological information measurement apparatus, and the communication unit collectively transmits the measurement values to the other apparatus.

2. The biological information measurement apparatus according to claim 1,
wherein when the accepting unit accepts the instruction from the user, the control unit causes all of the measurement values for the user and the subjects other than the user stored in the storage unit to be transmitted to the other apparatus, and deletes the measurement values transmitted by the communication unit from the storage unit in response to a notification from the other apparatus that has received the measurement values.

3. The biological information measurement apparatus according to claim 1,
wherein the measurement values stored in the storage unit are each further associated with information distinguishing whether or not the measurement value has already been transmitted by the communication unit, and
when the accepting unit accepts the instruction from the user, the control unit transmits a measurement value classified as a measurement value that has not yet been transmitted by the communication unit, of the measurement values for the user and the subjects other than the user stored in the storage unit, to the other apparatus.

4. The biological information measurement apparatus according to claim 3,
wherein in response to a notification from the other apparatus that has received the measurement value, the control unit updates the information associated with the measurement value in the storage unit and distinguishing whether or not the measurement value has already been transmitted.

5. The biological information measurement apparatus according to claim 1,
wherein the accepting unit includes a selection unit for selecting whether to collectively transmit measurement values for all of the plurality of subjects stored in the storage unit including the user or to designate, for each of the subjects other than the user of the plurality of subjects, whether or not to collectively transmit measurement values for the subject.

6. A biological information management apparatus for managing biological information measured on a biological information measurement apparatus, comprising:
a communication unit for receiving a measurement value from the biological information measurement apparatus, the measurement value being associated with information designating a storage region of the measurement value in the biological information measurement apparatus and characteristic information of the biological information measurement apparatus;
a storage unit for storing a correspondence relationship between a region in a storage apparatus and a combination of the information designating a storage region in the biological information measurement apparatus and the characteristic information of the biological information measurement apparatus; and
a control unit for storing the measurement value in the region in the storage apparatus specified from the combination of the information designating a storage region and the characteristic information of the biological information measurement apparatus,
wherein when a plurality of measurement values associated with combinations of the information designating different storage regions and the characteristic information of the biological information measurement apparatus are received, the control unit stores each of the plurality of measurement values in the region in the storage apparatus specified by the combination associated with the measurement value.

7. The biological information management apparatus according to claim 6, further comprising a first notification unit for issuing, when the control unit stores the measurement value, a notification to that effect.

8. The biological information management apparatus according to claim 6, further comprising a second notification unit for issuing, if the control unit cannot specify a region in the storage apparatus from the combination associated with the measurement value, a notification to that effect.

9. The biological information management apparatus according to claim 6, further comprising an updating unit for updating the correspondence relationship between a region in the storage apparatus and a combination of the information designating a storage region in the biological information measurement apparatus and the characteristic information of the biological information measurement apparatus, the correspondence relationship being stored in the storage unit.

10. The biological information management apparatus according to claim 6,
wherein the storage unit further stores a correspondence relationship between a subject and a region in the storage apparatus,
the biological information management apparatus further comprises an accepting unit for accepting a viewing request with respect to a measurement value for the subject, and
the control unit further executes a process for reading out the requested measurement value for the subject from the region in the storage apparatus corresponding to the subject and transmitting the measurement value.

11. The biological information management apparatus according to claim 10,
wherein the storage unit stores information for each subject, the information specifying a user who is permitted to view a measurement value for the subject, and
if it is determined, based on information specifying the user contained in the viewing request, that the user is permitted to view a measurement value for the subject, the control unit transmits the measurement value for the subject.

12. The biological information management apparatus according to claim 6,
wherein the storage unit further stores a correspondence relationship between a subject and a region in the storage apparatus, and
the biological information management apparatus further comprises a third notification unit for issuing, when the control unit specifies a subject with respect to whom a period of time that has elapsed from time and date of measurement of a latest measurement value stored to the present time is longer than a predefined threshold, a notification to that effect to a destination associated with the specified subject.

13. A biological information management system comprising a biological information measurement apparatus and a biological information management apparatus,
the biological information measurement apparatus being capable of measuring biological information on a plurality of subjects and storing the biological information on a subject-by-subject basis, and comprising:
a designation unit for designating a subject;
a measurement unit for measuring biological information on the subject;
a storage unit for storing the subject and a measurement value obtained by the measurement unit in association with each other;
an accepting unit for accepting an instruction to transmit a measurement value stored in the storage unit;
a communication unit for transmitting a measurement value stored in the storage unit to the biological information management apparatus; and
a control unit for performing a process in which when the accepting unit accepts an instruction from a user designated as one of the plurality of subjects to transmit a measurement value associated with the user, the measurement value for the user and measurement values for subjects other than the user of the plurality of subjects stored in the storage unit are each associated with information corresponding to the subject whom the measurement value is associated with and information characteristic of the biological information measurement apparatus, and the communication unit collectively transmits the measurement values to the biological information management apparatus, and
the biological information management apparatus being a biological information management apparatus for managing biological information measured on the biological information measurement apparatus, and comprising:
a communication unit for receiving a measurement value from the biological information measurement apparatus, the measurement value being associated with information, as the information corresponding to the subject, designating a storage region of the measurement value in the biological information measurement apparatus and the characteristic information of the biological information measurement apparatus;
a storage unit for storing a correspondence relationship between a region in a storage apparatus and a combination of the information designating a storage region and the characteristic information of the biological information measurement apparatus; and
a control unit for storing the measurement value in the region in the storage apparatus specified from the combination of the information designating a storage region and the characteristic information of the biological information measurement apparatus, and
when a plurality of measurement values associated with combinations of the information designating different storage regions and the characteristic information of the biological information measurement apparatus are received from the biological information measurement apparatus, the control unit of the biological information management apparatus stores each of the plurality of measurement values in the region in the storage apparatus specified by the combination associated with the measurement value.

14. A program for causing a computer to execute a process for managing biological information measured on a biological information measurement apparatus, the process comprising the steps of:
receiving a measurement value from the biological information measurement apparatus, the measurement value being associated with information designating a storage region of the measurement value in the biological information measurement apparatus and characteristic information of the biological information measurement apparatus;
specifying a region in a storage apparatus from a combination of the information designating a storage region in the biological information measurement apparatus and the characteristic information of the biological information measurement apparatus; and
storing the measurement value in the specified region in the storage apparatus,
wherein when a plurality of measurement values associated with combinations of the information designating different storage regions and the characteristic information of the biological information measurement apparatus have been received from the biological information measurement apparatus in the measurement value receiving step, each of the plurality of measurement values is stored in the region in the storage apparatus specified by the combination associated with the measurement value in the storing step.

15. A method of managing a measurement value obtained by a biological information measurement apparatus by storing the measurement value in a storage apparatus, the biological information measurement apparatus being capable of measuring biological information on a plurality of subjects and storing the biological information on a subject-by-subject basis, the method comprising:
a step in which the biological information measurement apparatus accepts an instruction to transmit a stored measurement value;
a step in which when an instruction from a user designated as one of the purality of subjects stored in the biological information measurement apparatus to transmit a measurement value associated with the user is accepted, the measurement value for the user and measurement values for subjects other than the user of the plurality of subjects stored in the biological information measurement apparatus are each associated with information designating a storage region in the biological information measurement apparatus, the storage region corresponding to the subject associated with the measurement value, and information characteristic of the biological information measurement apparatus, and the measurement values are collectively transmitted;
a step in which for each of the plurality of measurement values transmitted from the biological information measurement apparatus and associated with combinations of the information designating different storage regions and the characteristic information of the biological information measurement apparatus, a region in the storage apparatus is specified from the combination associated with the measurement value; and
a step in which each of the plurality of measurement values is stored in the region in the storage apparatus specified by the combination associated therewith.
